(19) United States
(12) Patent Application Publication
(10) Pub. No.: US 2026/0157722 A1
(43) Pub. Date: Jun. 11, 2026

(54) ULTRASOUND MOTION TRACKING TO EVALUATE RENAL FUNCTIONALITY

(71) Applicant: REGENTS OF THE UNIVERSITY OF MICHIGAN, Ann Arbor, MI (US)

(72) Inventors: Kourosh Kalayeh, Ann Arbor, MI (US); J. Brian Fowlkes, Ann Arbor, MI (US); William W. Schultz, Ann Arbor, MI (US); James A. Ashton-Miller, Ann Arbor, MI (US); John O. Delancey, Ann Arbor, MI (US); Ambani Sapan, Ann Arbor, MI (US); Maggie Zhang, Ann Arbor, MI (US); Bryan Sack, Ann Arbor, MI (US)

(21) Appl. No.: 19/073,254

(22) Filed: Mar. 7, 2025

Related U.S. Application Data
(60) Provisional application No. 63/573,410, filed on Apr. 2, 2024.

(51) Int. Cl. A61B 8/08; A61B 8/00; G01S 15/66; G01S 15/89; G06T 7/11; G06T 7/73

(52) U.S. Cl. CPC A61B 8/08; A61B 8/469; A61B 8/5207; G01S 15/66; G01S 15/8977; G06T 7/11; G06T 7/74; G06T 2200/24; G06T 2207/10132; G06T 2207/20104; G06T 2207/30084

(57) ABSTRACT

A method of performing ultrasound motion tracking for determining renal functionality includes obtaining a first and second ultrasound image at different times using an ultrasound probe. The method then identifies a reference region having a reference element at a first position in the first image and at a second position in the second image. The method further identifies regions of interest at first and second positions in the first and second images, the regions of interest including one or more target elements. The processor determines a change in position of the reference element and the regions of interest from the respective first and second positions of the reference region and regions of interest. The processor then determines respective time dependent ultrasound signal intensity values for each of the regions of interest in the first and second ultrasound images, and further determines renal functionality from the time dependent ultrasound signal intensity curve.

### CROSS-REFERENCE TO RELATED APPLICATIONS

This application claims the benefit of U.S. Provisional Application No. 63/573,410 filed Apr. 2, 2024, the entire contents of which are incorporated herein by reference in their entirety.

### FIELD OF THE DISCLOSURE

The invention generally relates to methods and systems for ultrasound tracking, and more particularly, for ultrasound tracking of renal function.

### BACKGROUND

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

Monitoring and tracking of the motion and functions of internal organs is used across a range of applications such as in surgery, medical diagnostics, disease monitoring and diagnosis, fertility tracking and monitoring, and performing biological and psychological research among other applications. Ultrasound imaging is a common modality for performing imaging of organs and tissues in a patient. Ultrasound imaging typically requires an expert to physically manipulate a probe or sensor to properly capture images of target tissues and organs. Moving the ultrasound probe or sensor according to real time movements of organs of a patient is often difficult and can result in capturing images that are not usable, or prevent adequate images for observing the motion of an organ or tissues over time. Inaccurate or unusable images further prevent the use of ultrasound imaging for tracking organ function for further diagnosis and treatment of diseases and conditions.

Monitoring kidney function is required for diagnosis and treatment of many diseases and conditions. Kidney obstruction is a common and morbid condition in pediatric and adult patients caused by acquired or congenital anatomic abnormalities of the urinary drainage system. Consequences of kidney obstruction include pain, infection, temporary kidney dysfunction, and permanent kidney damage. Current radiology tests to diagnose and assess obstruction include cross-sectional imaging (e.g. CT or MRI) and nuclear medicine renal scans. For one example, monitoring kidney functionality is used in diagnosing ureteropelvic junction obstruction (UPJO) and determining its treatment. Typical imaging modalities, are insufficient in providing monitoring and tracking of kidney functionality for UPJO treatment. Typically imaging studies may allow for imaging of a dilated and urine filled kidney, thinning of the kidney tissue, slow drainage of urine from the kidney, and unrecoverable lost kidney function. Unfortunately, the presence or absence of these findings often does not correlate with kidney obstruction. Additionally, patients, organs, or tissues may move during imaging which renders multiple frames useless in further analysis for determining kidney functionality. One such complicating challenge in determining kidney function is due to kidney movement during respiratory motion. This often results in imaged regions falling out of frame or causes noisy data that is not usable in any meaningful determination of organ function. Therefore, improved systems and methods are desirable for monitoring kidney functionality for diagnosis and treatment of various conditions.

### SUMMARY OF THE INVENTION

Techniques and systems are provided for performing ultrasound motion tracking and monitoring of renal functionality. In an implementation the method includes obtaining, by an ultrasound sensor, a plurality of ultrasound images of a region of tissue of a patient. The plurality of images includes a first ultrasound image and a second ultrasound image, the second ultrasound image being obtained at a different time than the first ultrasound image. A processor or user identifies a reference region at a first position in the first ultrasound image with the reference region including a reference element. A processor or user then identifies one or more regions of interest in the first ultrasound image with the one or more regions of interest including one or more target elements. The one or more regions of interest each have respective first positions defined relative to the reference region in the first ultrasound image. The processor then determines a second position of the reference region in the second image of the plurality of images, and a change in position of the reference region from the first position and the second position of the reference region. The reference region may be loosely or generally around a center of a kidney. The processor further determines a second position of each of the regions of interest in the second ultrasound image from the change in position of the reference region, and then determines respective ultrasound signal intensity values for each of the one or more regions of interest in the first and second ultrasound images. The processor generates a time dependent ultrasound signal intensity curve from the respective ultrasound signal intensity values, and then determines renal functionality from the time dependent ultrasound signal intensity curve.

In examples, the method may further include filtering, by the processor, the signal intensity values for each of the one or more regions of interest according to one or more intensity thresholds and generating a filtered set of ultrasound signal intensity values, and wherein generating the time dependent ultrasound signal intensity curve comprises determining the time dependent ultrasound intensity curve from the filtered set of ultrasound signal intensity values.

In further examples, the determining the renal function from the time dependent ultrasound signal intensity curve comprises determining one or more of a decay time of the curve, mean transient time (MTT), time to peak (TPP), full width at half maximum (FWHM), peak intensity (PI), area under the curve (AUC), wash in time (WIN), and wash out time (WOT).

In yet more examples, the method may include averaging, by the processor, the determined ultrasound signal intensity values to generate an averaged time dependent ultrasound signal intensity curve, and determining, by the processor, the renal functionality from the averaged time dependent ultrasound signal intensity curve. The reference element may include a portion of a kidney such as a renal sinus, and the target element may include any portion of a kidney such as a renal sinus, renal pelvis, renal parenchyma, region of blood vessels, or other tissue or structure in the renal region.

In another implementation, disclosed is a system for performing ultrasound motion tracking and monitoring of renal functionality. The system includes an ultrasound detector, a processor configured to execute machine readable instructions, and a non-transitory computer-readable memory having machine readable instructions stored thereon. When executed by the processor, the machine readable instructions cause the system to (a) obtain, by an ultrasound sensor, a plurality of ultrasound images of a region of tissue of a patient, the plurality of images including a first ultrasound image and a second ultrasound image, the second ultrasound image obtained at a different time than the first ultrasound image, (b) identify a reference region at a first position in the first ultrasound image, the reference region including a reference element, (c) identify one or more regions of interest in the first ultrasound image, the one or more regions of interest including one or more target elements, the one or more regions of interest each having respective first positions relative to the reference region in the first ultrasound image, (d) determine a second position of the reference region in the second image of the plurality of images, (e) determine a change in position of the reference region from the first position and the second position of the reference region, (f) determine a second position of each of the regions of interest in the second ultrasound image from the change in position of the reference region, (g) determine respective ultrasound signal intensity values for each of the one or more regions of interest in the first and second ultrasound images, (h) generate a time dependent ultrasound signal intensity curve from the respective ultrasound signal intensity values, and (i) determine renal functionality from the time dependent ultrasound signal intensity curve.

In examples, the machine readable instructions may further cause the system to filter, by the processor, the signal intensity values for each of the one or more regions of interest according to one or more intensity thresholds and generate a filtered set of ultrasound signal intensity values, and wherein to generate the time dependent ultrasound signal intensity curve, the machine readable instructions cause the system to determine the time dependent ultrasound intensity curve from the filtered set of ultrasound signal intensity values.

In further examples, to determine the renal function from the time dependent ultrasound signal intensity curve, the machine readable instruction cause the system to determine one or more of a decay time of the curve, mean transient time (MTT), time to peak (TPP), full width at half maximum (FWHM), peak intensity (PI), area under the curve (AUC), wash in time (WIN), and wash out time (WOT).

In yet more examples, the machine readable instructions may cause the system to average the determined ultrasound signal intensity values to generate an averaged time dependent ultrasound signal intensity curve, and determine the renal functionality from the averaged time dependent ultrasound signal intensity curve. The reference element may include a portion of a kidney such as a portion of a renal sinus, and the target element may include a portion of a kidney.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 is a schematic illustration of an example scenario for performing ultrasound tracking of tissues and organs.

FIG. 2 is a block diagram of an example system for performing the ultrasound image and organ motion tracking methods described herein.

FIG. 3 is an example sagittal B-mode ultrasound image of a renal region of a patient taken according to the scenario of FIG. 1, and the system of FIG. 2.

FIG. 4 is a flow diagram of a method for performing ultrasound motion tracking of an organ or tissue, such as of a kidney, for determining renal functionality.

FIG. 5A is an example ultrasound image of a renal region of a subject as may be obtained by the ultrasound probe.

FIG. 5B is an ultrasound image of the renal region of a patient taken at a different time than the ultrasound image of FIG. 5A.

FIG. 6A is a plot of ultrasound signal time intensity curve and associated fits for a kidney exhibiting normal kidney function.

FIG. 6B is a plot of ultrasound signal time intensity curve and associated fits for a kidney exhibiting renal obstruction.

FIG. 6C is a bar graph showing a comparison of mean transit times for normal and obstructed kidneys for five subjects.

FIG. 6D is a bar plot showing comparisons of mean transit times of renal processing of ultrasound contrast agent for normal renal function, and for patients before and after surgery for correcting renal obstruction.

### DETAILED DESCRIPTION

Provided are techniques for tracking tissues and organs using machine vision processes. The specific application described utilizes machine vision to identify and track organs and tissues in the renal region of a patient. The disclosed methods and systems enable automated analysis and tracking of organs for medical diagnosis and treatment, such as for treatment of ureteropelvic junction obstruction (UPJO) and evaluating outcome of pyeloplasty surgeries. Ultrasound imaging is used to image the renal region of a patient including a portion of a kidney (e.g., renal sinus, renal pelvis, veins, blood vessels, renal parenchyma, renal fat, etc.), and machine vision then automatically tracks the movement of tissues and organs in real-time. An ultrasound intensity is determined for various regions of interest and kidney functionality is determined from ultrasound signal intensity over time. Integrating computer vision with dynamic contrast-enhanced ultrasound (DCEUS) enables real-time, or near real-time monitoring and capturing of dynamic movements of organs such as the kidney and surrounding structures. Further, the disclosed methods allow for monitoring renal microcirculation between normal renal function and obstructed kidneys.

FIG. 1 is a schematic illustration of an example scenario 100 for performing ultrasound tracking of tissues and organs. In the example scenario 100 of FIG. 1, an ultrasound probe 120 is placed against the body 110 of a patient to provide ultrasound waves 112 to the body 110 and to detect reflected ultrasound vibrations. A gel, such as an ultrasound gel, may be applied to a portion of the body 110 and the ultrasound probe 120 may be placed against the portion of the body 110 having the ultrasound gel to increase the coupling of the ultrasound waves 112 into the body 110 of the patient. The ultrasound probe 120 may then receive reflected ultrasound vibrations to image tissues and organs inside of the body 110. In the illustrated example, the ultrasound probe 120 is positioned against the body 110 to image a renal region, including a portion of a kidney of the patient. The ultrasound probe may image one or more of a renal sinus 105, kidney 107, bladder 115a, and ureter 115b, among other tissues and/or organs.

As shown in FIG. 1. the ultrasound probe 120 is positioned on a side of the patient, in other examples the ultrasound probe 120 may be placed on the back of the patient, for performing ultrasound imaging. Movement of the ultrasound probe 120 during imaging also causes the positions of organs to change in the resultant ultrasound images, and may cause the kidney to go out of the imaging plane in certain ultrasound images or frames. Movement of the ultrasound probe 120 must be decoupled from the structural displacements and deformations of the organs during ultrasound imaging for performing motion tracking. To address this challenge, a portion of the kidney, and specifically in the disclosed examples, the renal sinus 105 is used as a stationary reference point. Therefore, as will be shown, movement of the kidney in the ultrasound images is tracked and monitored in relation to a coordinate system referenced to the renal sinus 105. This, in turn, enables the comparison of movements of structures adjacent to the renal sinus 105, and allows for the contextual registration of the movements or nearby organs and tissues. This further allows for monitoring of ultrasound signal intensity from a plurality of regions of interest over time, and further to determine renal functionality from the ultrasound signal intensity over time.

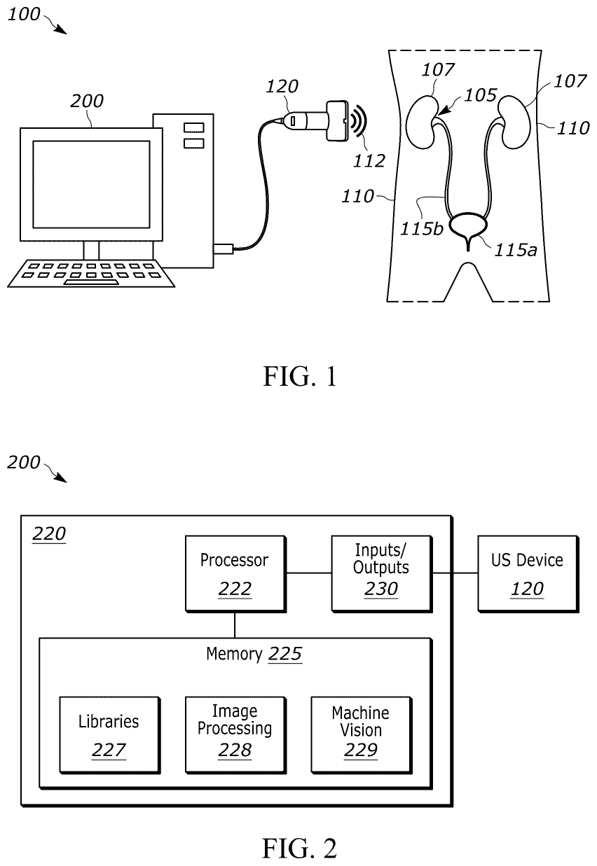

The ultrasound probe 120 generates a signal indicative of the detected reflected ultrasound waves and provides the signal to one or more systems 200 for performing image processing, machine vision operations, and for displaying one or more ultrasound images and videos. FIG. 2 is a block diagram of an example system 200 for performing the ultrasound image and organ motion tracking methods described herein. The system 200 may be used to perform any of the methods disclosed herein. The system 200 includes one or more processors 222 that execute machine readable instructions for performing motion tracking according to the described methods. The processor may access one or more memories 225 to retrieve data, store data, and retrieve or store machine readable instructions. The one or more memories 225 may store one or more software or language libraries 227 (e.g., Python open source libraries, or other software and processing libraries), image processing algorithms 228, and machine vision processes 229. The image processing algorithms 228 may include image manipulations and transforms, filtering and noise reduction, grayscale conversion, histogram equalization, thresholding, background subtraction feature detection, corner detection based algorithms, tracking algorithms (e.g., optical flow based algorithms), etc. The machine vision processes 229 may include one or more motion tracking algorithms, for example, optical flow based algorithms.

The system 200 further includes one or more input/output ports 230 or devices. For example, the input/output ports 230 may include wired or wireless communication channels that receive or provide data to external networks, servers, and devices. The input/output ports 230 may include display devices such as monitors and touchscreens to provide images and video to a user. The input/output ports 230 may further include input devices such as one or more keyboards, mice, touchscreens, etc. In the example provided, the probe 120 provides data indicative of ultrasound images and video to the system 200 via the input/output ports 230.

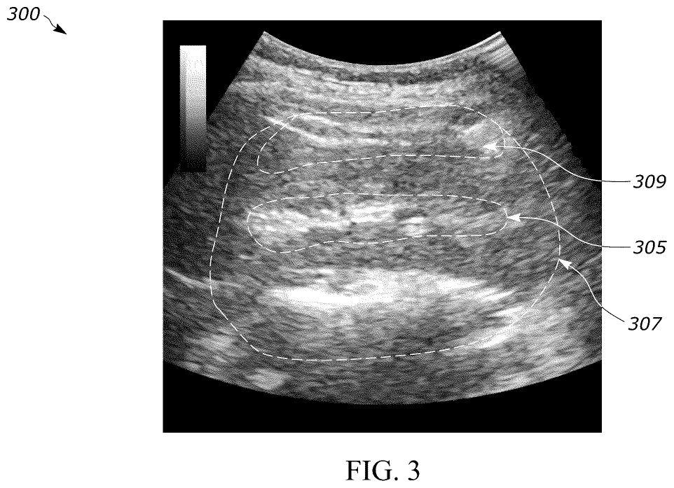

FIG. 3 is an example B-mode ultrasound image 300 of the renal region of a patient taken according to the scenario 100 of FIG. 1, and the system 100 of FIG. 2. The image includes a kidney 307 including a renal sinus 305 and parenchyma 309. The renal sinus 307 may include a renal pelvis, renal fat, renal blood vessels, and other tissues in the renal region. In examples described further herein, the renal sinus 305 or portions of the renal sinus 305, are used as a reference to track the movement of target regions of functional renal parenchyma to monitor ultrasound signal intensity from contrast enhanced images of the target regions overtime.

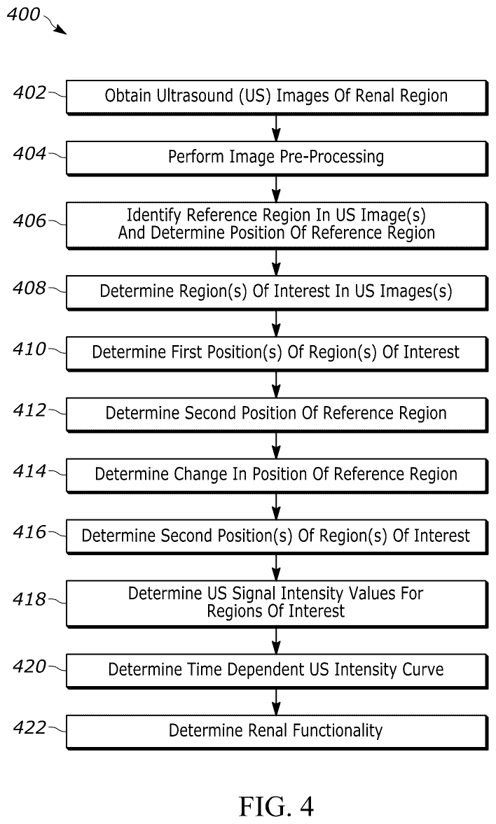

FIG. 4 is a flow diagram of a method 400 for performing ultrasound motion tracking of an organ or tissue, such as of a kidney, for determining renal functionality. The method 400 may be performed using the system 200 illustrated in FIGS. 1 and 2, and as such, the method 400 will be described with continued reference to elements of FIGS. 1 and 2 for clarity. The method 400 includes obtaining a plurality of ultrasound images of a region of tissue, such as a renal region including a portion of a kidney, of a patient, at block 402. The images may be obtained using an ultrasound probe, such as the probe 120, or by another ultrasound device such as a wearable device, tabletop device, etc. The plurality of images includes at least two images taken at different times to determine movement of a portion of the kidney, and to determine renal functionality from ultrasound signal intensity over time from the two different images. For simplicity, the method 400 is described herein with reference to only two ultrasound images, but it should be understood that the method 400 may be applied to any number of images for tracking the movement of a kidney. For example, the ultrasound probe 120 may obtain a series of images over time to track the motion of a kidney and to determine ultrasound signal intensity over time for specific portions of the kidney. The ultrasound probe may obtain images at a rate of 10 to 100 frames pers second, 100 to 1000 frames per second, or greater than 1000 frames per second. The method 400 may be used to track the motion and determine ultrasound signal intensity from a kidney over seconds, minutes, tens of minutes, or longer.

The processor may then identify ultrasound images wherein the portion of the kidney is not in focus or has moved out of the image frame. The processor may then remove the ultrasound images that do not contain a portion of the kidney, or a minimum amount of the kidney is imaged, from further processing. Removing ultrasound images without a portion of the kidney reduces the overall processing time as the ultrasound images without the portion of the kidney are not useful in determining renal function. Additionally, if the processor determines that a region of interest or portion of the kidney has moved out of frame, the processor may prompt the user to identify an additional region of interest, or a region of interest in the ultrasound image to replace the region that moved out of frame.

The one or more processors 222 may then perform image preprocessing on one or more of the ultrasound images at block 404. The image preprocessing may include performing one or more image transformations, greyscale conversion, histogram equalizations, contrast enhancement, brightness normalization, filtering and noise reduction, thresholding, and background subtraction. For example, the processor may first convert an ultrasound image to greyscale to reduce any color data to a single grey channel to simplify subsequent processing while retaining essential image data and information. A contrast limited adaptive histogram equalization (CLAHE) may then be applied to enhance image contrast. A CLAHE is typically effective in improving the visibility of features in medical imaging by normalizing the brightness and increasing contrast of medical images, such as ultrasound images.

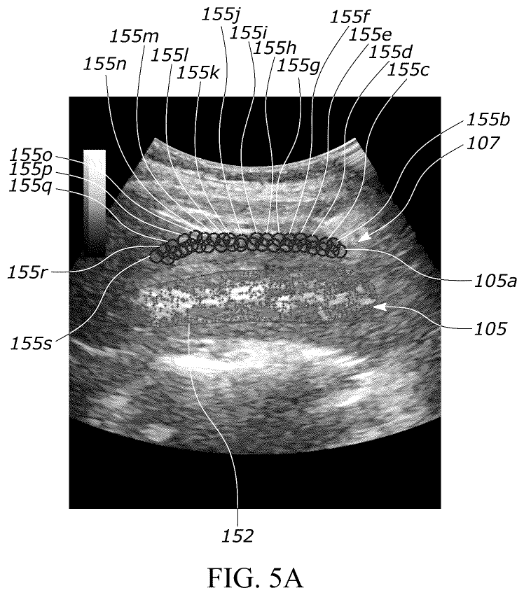

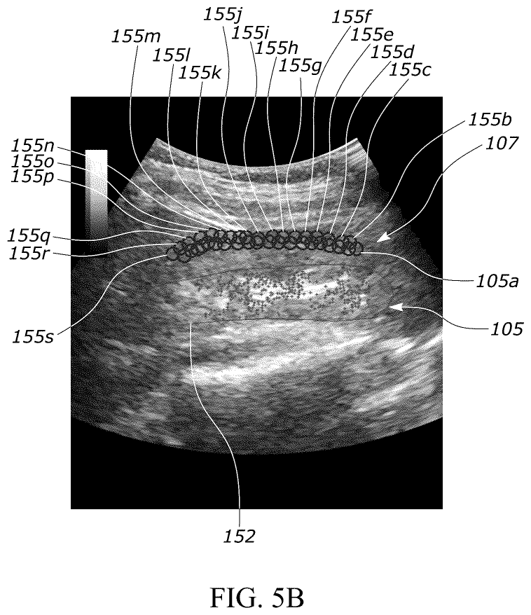

FIG. 5A is an example ultrasound image as may be obtained by the ultrasound probe 120. The image is of renal region including the renal sinus 105 and the kidney 107. At block 406 a reference region is identified in the first ultrasound image. FIG. 5A shows a reference region 152 being an oblong region about an area of the image including the renal sinus 105, at a central region of the kidney. The reference region 152 includes one or more reference elements that are to be tracked, and used as a reference for other regions of interest in the ultrasound image, described further herein. In the illustrated example, the renal sinus 105 is used as the reference element and the reference region 152 is determined as a region including the renal sinus 105, or a portion of the renal sinus 105. As used herein, the word “portion” with reference to an anatomical structure includes at least part of the anatomical structure and may include the entire anatomical structure. The reference region 152 may be identified by a processor, such as the one or more processors 222, or the reference region may be identified by a user via one or more user inputs or devices of the input/output ports 230. For example, the system 200 may display the first ultrasound image, and a user may use a mouse or touch screen to indicate the reference region. In examples, the processor or a user identifies the reference element (e.g., renal sinus, renal pelvis, blood vessels, parenchyma etc.) and the processor 222 further defines the reference region 152 to contain the reference element or a portion of the reference element.

The processor 222 then determines a position of the reference region 152 in the first image. The processor 222 may determine the reference region 152 in the first image via performing image segmentation including one or more of, without limitation, edge detection, contrast analysis, thresholding, contouring, one or more morphological operations, or by applying another algorithm, machine vision, or imaging processing technique. The position may be determined based on pixel number, or by coordinates of the vertices of a polygon drawn by a user or determined by the processor. Additionally, the processor can determine the location of the reference region 152 with respect to a global coordinate system of the image. In examples, the processor 222 may take a global coordinate system as a Cartesian coordinate system defined with an origin at a corner of an ultrasound image such as the top left or bottom left corner, or pixel, of the image. Further, the processor may further determine a length and width of the rectangular reference region 152 and an angular orientation of the reference region in the image from the various coordinates of the vertices of the reference region.

At block 408 the method 400 includes determining one or more regions of interest in the first ultrasound image. The one or more regions of interest include at least one target element in the first image which may be a tissue or organ of the patient, such as a portion of a kidney. FIG. 5A illustrates an example ultrasound image with a plurality of regions of interest 155a-155s indicative of different portions of tissue of the kidney 107. Each of the regions of interest 155a-155s are illustrated as circles that include a portion of the kidney 107. In the specific example shown, the regions of interest each include an area of renal parenchyma which is functioning tissue of the kidney. An ultrasound contrast agent is used in the disclosed methods to observe ultrasound signals from the renal parenchyma to determine the renal functionality due to processing of the ultrasound contrast agent in the renal parenchyma. The regions of interest may be determined as portions of the kidney at locations relative to the position of the reference region about the reference element. In examples, a processor may perform image processing or machine vision processes to determine the regions of interest 155a-155s, or a user may provide a user input to determine the regions of interest 155a-155s and respective target elements. To determine the regions of interest, the processor 222 may perform image segmentation including one or more of, without limitation, edge detection, contrast analysis, thresholding, contouring, or one or more morphological operations in addition to other algorithms and image processing techniques. In the example provided, each of the different portions of the kidney 107 are used as the target elements for each respective region of interest 155a-155s. In a first image, such as the first ultrasound image, a user may be prompted to provide an input indicative of one or more regions of interest. Additionally, it should be understood that while the reference region 152 and the regions of interest 155a-155s are illustrated respectively as an oblong polygon and circles, the reference region 152 and the regions of interest 155a-155s may independently be any other shape indicative of respective reference elements and target elements for performing motion tracking of tissues or organs. For example, each of the reference region 152, and regions of interest 155-155s may independently be circles, ellipses, ovals, squares, triangles, polygons, asymmetric shapes, a freeform shape, or another two-dimensional geometric shape for indicating a region with a corresponding reference or target element. Circular regions of interest are implemented herein for simplicity and clarity of demonstration. The circular regions of interest maintain a consistent and simple description in position and orientation in a Cartesian coordinate system with respect to the reference region, as used herein.

The processor then determines first positions of each of the regions of interest 155a-155s in the first image at block 410. To determine positions of the regions of interest 155a-155s the processor may establish a coordinate axis or coordinate system based on the position and orientation of the reference region 152. In the provided examples, the processor determines the positions of the regions of interest 155a-155s in a coordinate system with origin based in the top left corner of the ultrasound image. The positions of each of the regions of interest 155a-155s may then be established as the center of each of the circular regions of interest 155a-155s in the established coordinate system 170. The positions of the regions of interest 155a-155s are determined relative to the reference region 152 to track the movement of target tissues in the regions of interest 155a-155s along with the movement of the reference element in the reference region 152. As described further, the ultrasound signal intensity of the regions of interest 155a-155s is tracked across multiple ultrasound images to determines ultrasound signal intensity overtime. As such, the regions of interest 155a-155s may be described as signal extraction regions, or signal-extraction regions of interest 155a-155s.

At block 412, the processor determines a second position of the reference region 152 in the second ultrasound image. FIG. 5B is an ultrasound image of the renal region of a patient taken at a different time than the ultrasound image of FIG. 5A. The processor determines the second position of the reference region 152, and moves the reference region 152 and signal-extraction regions of interest 155a-155s accordingly.

The processor 122 further determines a change in position of the reference region from the first and second positions of the reference region in the first and second images, respectively, at block 414. The change in position may be determined as a translation of the original or centroid of the oblong reference region illustrated in FIGS. 5A and 5B. In determining the change in position, the processor may determine a difference between the first position of the reference region 152 in the first ultrasound image, and the second position of the reference region 152 in the second ultrasound image in the global coordinate system of the ultrasound image. In implementation, the method 400 is performed on a plurality of ultrasound images including more than 2 ultrasound images, with the position and orientation of the reference region 152 being determine in each ultrasound image. In such examples, the processor 122 may determine a path of motion of the reference region across the plurality of ultrasound images and determine a corresponding position of the reference region 152 in each ultrasound image over time.

The processor then determines the positions of the regions of interest 155a-155s in the second ultrasound image by shifting the regions of interest from the first image to the second image depending on the determined movement or translation of the reference region 152 from the first image to the second image. As such, the processor tracks the movement of the tissues in the regions of interest 155a-15m across images according to the movement of the reference region 152 between respective images. Therefore, the second positions of the regions of interest 155a-155s are further determined, or estimated, using the change in position of the reference region 152 from the first position to the second position of the reference region 152.

As previously described, the positions of the reference region 152 and the regions of interest 155a-155s may be determined for a plurality of ultrasound images, and respective region of interest paths may be determined based on the positions of the regions of interest in each ultrasound image. To determine the paths of the reference region 152, and the regions of interest 155a-155s the processor may determine a geometric transformation matrix between the first and second ultrasound images, or generally between consecutive frames of a plurality of ultrasound images, for each region of interest 155a-155s and the reference region 152. One such example transformation matrix is a similarity transformation matrix given by,

$$M = [ \begin{matrix}s \cdot cos θ & - s \cdot sin θ & {t}_{x} \\ s \cdot sin θ & s \cdot cos θ & {t}_{y}\end{matrix} ] . \qquad EQ 1$$

The provided similarity transform matrix has four degrees of freedom; two translational tₓ and t_{y} one rotational θ, and a scaling factor s. The similarity transform matrix maintains the shape of an object in an image which preserves the angles of a region of interest. In determining the transformation matric between two images, an OpenCV algorithm such as estimateAffinePartial2D may be implemented to determine the change in position of the reference region between the first and second ultrasound images. Once the transformation matrix is determined, the processor then applies the transformation matrix to each of the regions of interest 155a-155s, applied at the center of the regions of interest 155a-155s to determine the motion or paths of regions of interest 155a-155s from image to image.

The processor 122 then determines respective ultrasound signal intensity values for each of the regions of interest 155a-155s in the first and second ultrasound images, at block 418. The processor 122 may average pixel intensities in each region of interest 155a-155s and determine an averaged ultrasound signal intensity for each region of interest 155a-155s for the first and second ultrasound images, or the processor may average the ultrasound signal intensity across all regions of interest 155a-155s and determine a single ultrasound signal intensity value for each of the first ultrasound image, and the second ultrasound image. In practice, more than two ultrasound images may be obtained, and respective ultrasound signal intensities may be determined using the regions of interest of each independent ultrasound image.

In examples, some regions of interest 155a-155s may overlap with or move to a dark region of tissue, or include a region of tissue outside of the kidney. Therefore, to reduce the potential for these types of regions of interest from contaminating the measurement, or from causing error in the measurement and analysis for renal function, the processor may only use regions of interest within certain percentile of ultrasound signal intensities, for example pixel intensities within 10 and 90 percentile, withing 25 and 75 percentile, etc., across all regions of interest for a given image. This reduces the overall noise and may increase the accuracy of monitoring renal function and determining potential kidney obstruction.

In typical ultrasound imaging, the intensities of ultrasound echoes from different targets are often assigned colors according to a colormap indicative of the intensity and strength of reflected ultrasound echoes from tissues. This ultrasound reflected signal color map is usually non-linear to optimize the representation of image information across a wide variable color range. This is considered to present the image information more accurately as the human eye is more sensitive to changes in certain color intensity ranges. In determining the ultrasound signal intensity values, the processor may determine a linear pixel intensity value, Iₚₓ, from the non-linear color map bases ultrasound signal intensity values. The processor then may convert the linear pixel intensity values, Iₚₓ, to a logarithmic scale in dB from the acquisition dynamic range of the ultrasound probe and system, such that

$${I}_{dB} = \frac{{I}_{px}}{255} \cdot DR + G \qquad EQ . 2$$

where G is a gain value applied to the ultrasound signal. The processor may then further log-decompress the decibel signal intensity values. The log-decompression is performed to provide a linear relationship between ultrasound signal intensity and concentration of contrast agents flowing in and out of the kidney or renal region. This may be performed in implementations where raw ultrasound data is not readily available and logarithmic scale data must be converted. At block 420 the processor then determines a time dependent ultrasound signal intensity curve from the respective ultrasound signal intensity values of the first and second images. The time dependent curve is determined as the change in ultrasound signal intensity over time. The time dependent ultrasound signal intensity curve may be determined for each ROI 155a-155s independently, or a single curve may be determined from the average ultrasound signal intensity value across all regions of interest for each given ultrasound image.

The processor 122 then determines renal functionality from the time dependent ultrasound signal intensity curve at block 422. The processor 122 may determine renal function (e.g., obstruction, normal functionality, abnormal functionality, etc.) from one or more of a decay time of the ultrasound signal intensity curve, mean transient time (MTT), time to peak (TPP), full width at half maximum (FWHM), peak intensity (PI), area under the curve (AUC), wash in time (WIN), and wash out time (WOT). For example, one potential indication of abnormal renal functionality, is a restriction in blood flow, which can be identified by monitoring circulation of ultrasound contrast agent in the blood stream.

The method 400 was demonstrated clinically on five subjects aged 25 to 73 years with a mean age of 48 years. Four of the subjects exhibited a left kidney obstruction, and one subject had a right kidney obstruction. Clinical GE LOGIQ® E9 and E10 ultrasound scanners, equipped with C1-6 and C2-9 probes, were used for DCEUS imaging of the kidneys. DEFINITY® ultrasound contrast microbubbles were administered intravenously at a dose of 0.3 mL per kidney (within the FDA guidelines). Each microbubble bolus injection was followed by a 10 ml saline flush. Ultrasound cine loops of the obstructed and normal kidneys were initiated shortly after the saline flush and were continued for at least 80 s thereafter. The cine loops were stored for offline analysis.

To accurately analyze contrast flow dynamics, it was critical to account for respiratory motion of the kidney. The method 400 of FIG. 4 was performed using custom Python-based tracking software. At least 10 circular regions of interest were used, each with a radius of 5 mm identified on the renal cortex to extract pixel intensities. The pixel intensities were transformed into log-decompressed ultrasound signal intensities, which were then averaged across all regions of interest. The average intensities were filtered using robust locally-weighted scatterplot smoothing and fitted to a lognormal distribution.

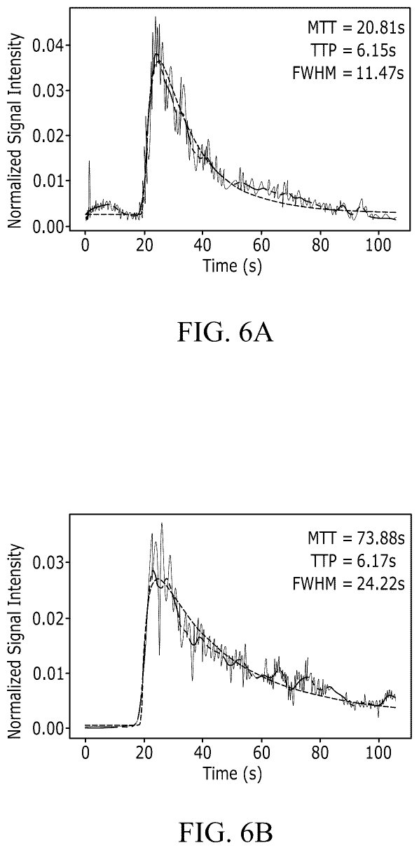

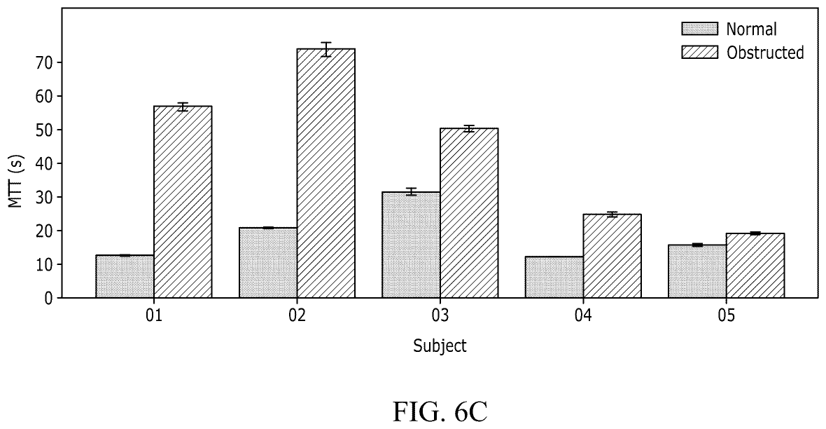

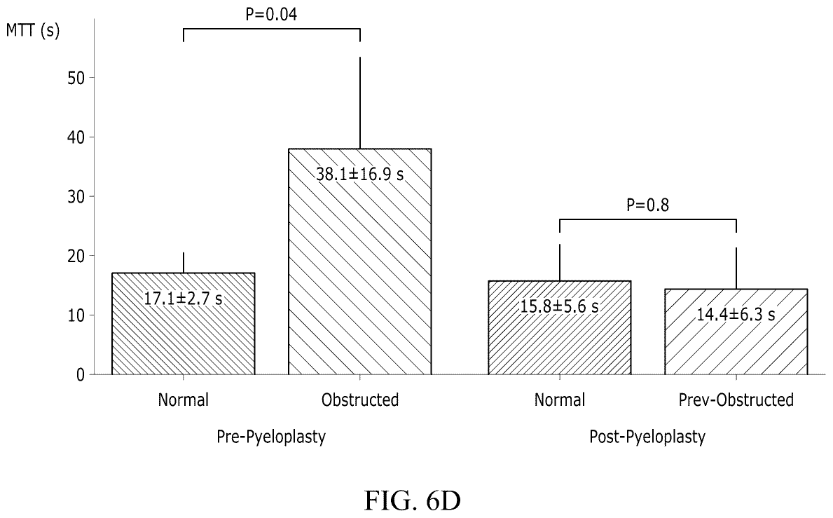

FIGS. 6A and 6B are plots of ultrasound signal time intensity curve and associated fits for a kidney exhibiting normal kidney function, and obstructed functionality respectively. The plots of FIGS. 6A and 6B report time to peak (TTP) values, full width at half max (FWHM) values, and mean transit times (MTT), as examples, to compare the normal and obstructed renal functionality. The MTT shows the largest difference between the normal and obstructed kidney functionalities with the MTT being over three times longer for the obstructed kidney than for the normal kidney. FIG. 6C is a bar graph showing a comparison of the MTT for normal and obstructed kidneys for the five subjects. The mean MTT for normal functionality was 17.1±2.7 s, and the mean MTT for obstructed kidney was 38.1±16.9 s (statistically significant with p=0.04).

FIG. 6D is a bar plot showing comparisons of mean transit times of renal processing of ultrasound contrast agent for normal renal function, and for patients before and after surgery for correcting renal obstruction. The MTT values presented in FIG. 6D were obtained using the disclosed systems and methods. Each patient had one kidney with normal functionality, and one obstructed kidney, as presented by the data of FIG. 6D. It was found that the kidneys with normal renal functionality exhibited MTT values of 17.1±2.7 s and 15.8±5.6 s before and after pyeloplasty surgery, which is expected as the normal kidney functionality should not change for the non-obstructed kidney. The kidneys having renal obstruction showed an average MTT of 38.1±16.9 s before surgery, which was reduced to 14.4±6.3 s after pyeloplasty, showing that the previously obstructed kidneys now exhibited normal renal functionality after surgery.

### Additional Aspects

Aspect 1. A method for performing ultrasound tracking to determine renal functionality, the method comprising: obtaining, by an ultrasound sensor, a plurality of ultrasound images of a region of tissue of a patient, the plurality of images including a first ultrasound image and a second ultrasound image, the second ultrasound image obtained at a different time than the first ultrasound image; identifying a reference region at a first position in the first ultrasound image, the reference region including a reference element; identifying, one or more regions of interest in the first ultrasound image, the one or more regions of interest including one or more target elements, the one or more regions of interest each having respective first positions defined relative to the reference region in the first ultrasound image; determining, by the processor, a second position of the reference region in the second image of the plurality of images; determining, by the processor, a change in position of the reference region from the first position and the second position of the reference region; determining, by the processor, a second position of each of the regions of interest in the second ultrasound image from the change in position of the reference region; determining, by the processor, respective ultrasound signal intensity values for each of the one or more regions of interest in the first and second ultrasound images; generating, via the processor, a time dependent ultrasound signal intensity curve from the respective ultrasound signal intensity values; and, determining renal functionality from the time dependent ultrasound signal intensity curve.

Aspect 2. The method of Aspect 1, further comprising filtering, by the processor, the signal intensity values for each of the one or more regions of interest according to one or more intensity thresholds and generating a filtered set of ultrasound signal intensity values, and wherein generating the time dependent ultrasound signal intensity curve comprises determining the time dependent ultrasound intensity curve from the filtered set of ultrasound signal intensity values.

Aspect 3. The method of either Aspect 1 or Aspect 2, wherein determining the renal function from the time dependent ultrasound signal intensity curve comprises determining one or more of a decay time of the curve, mean transient time (MTT), time to peak (TPP), full width at half maximum (FWHM), peak intensity (PI), area under the curve (AUC), wash in time (WIN), and wash out time (WOT).

Aspect 4. The method of any of Aspects 1 to 4, further comprising: averaging, by the processor, the determined ultrasound signal intensity values to generate an averaged time dependent ultrasound signal intensity curve; and determining, by the processor, the renal functionality from the averaged time dependent ultrasound signal intensity curve.

Aspect 5. The method of any of Aspects 1 to 4, wherein the reference element comprises a portion of a kidney.

Aspect 6. The method of any of Aspects 1, wherein the target element comprises a portion of a kidney.

Aspect 7. The method of claim 1, further comprising performing, by the processor, pre-processing on at least one image of the first ultrasound image and second ultrasound image, the preprocessing including one or more of a grayscale conversion, blurring, spatial frequency filtering, a histogram equalization, a background subtraction, a contrast enhancement, a brightness normalization, filtering and noise reduction, grayscale conversion, histogram equalization, thresholding, background subtraction feature detection, corner detection based algorithms, tracking algorithms (e.g., optical flow based algorithms), etc.

Aspect 8. The method of any of Aspects 1 to 7, wherein identifying the reference region comprises receiving, via a user interface, a user identification of the reference region.

Aspect 9. The method of any of Aspects 1 to 8, wherein identifying the reference region comprises identifying, via the processor, the reference region via image segmentation.

Aspect 10. The method of any of Aspects 1 to 9, wherein identifying the one or more regions of interest comprises receiving, via a user interface, a user identification of the one or more regions of interest.

Aspect 11. The method of any of Aspects 1 to 10, wherein identifying the reference region comprises identifying, via the processor, the one or more regions of interest via image segmentation.

Aspect 12. A system for performing ultrasound tracking, the system comprising: an ultrasound detector; a processor configured to execute machine readable instructions; and a non-transitory computer-readable memory having machine readable instructions stored thereon, that when executed by the processor, cause the system to: obtain, by an ultrasound sensor, a plurality of ultrasound images of a region of tissue of a patient, the plurality of images including a first ultrasound image and a second ultrasound image, the second ultrasound image obtained at a different time than the first ultrasound image; identify a reference region at a first position in the first ultrasound image, the reference region including a reference element; identify one or more regions of interest in the first ultrasound image, the one or more regions of interest including one or more target elements, the one or more regions of interest each having respective first positions relative to the reference region in the first ultrasound image; determine a second position of the reference region in the second image of the plurality of images; determine a change in position of the reference region from the first position and the second position of the reference region; determine a second position of each of the regions of interest in the second ultrasound image from the change in position of the reference region; determine respective ultrasound signal intensity values for each of the one or more regions of interest in the first and second ultrasound images; generate a time dependent ultrasound signal intensity curve from the respective ultrasound signal intensity values; and determine renal functionality from the time dependent ultrasound signal intensity curve.

Aspect 12. The system of Aspect 11, wherein the machine readable instructions further cause the system to filter the signal intensity values for each of the one or more regions of interest according to one or more intensity thresholds and generate a filtered set of ultrasound signal intensity values, and wherein to generate the time dependent ultrasound signal intensity curve, the machine readable instructions cause the system to determine the time dependent ultrasound intensity curve from the filtered set of ultrasound signal intensity values.

Aspect 13. The system of either Aspect 11 or 12, wherein to determine the renal function from the time dependent ultrasound signal intensity curve, the machine readable instructions cause the system to determine one or more of a decay time of the curve, mean transient time (MTT), time to peak (TPP), full width at half maximum (FWHM), peak intensity (PI), area under the curve (AUC), wash in time (WIN), and wash out time (WOT).

Aspect 14. The system of any of Aspects 11 to 13, wherein the machine readable instructions further cause the system to: average the determined ultrasound signal intensity values to generate an averaged time dependent ultrasound signal intensity curve, and; determine the renal functionality from the averaged time dependent ultrasound signal intensity curve.

Aspect 15. The system of any of Aspects 11 to 14, wherein the reference element comprises a portion of a kidney.

Aspect 16. The system of any of Aspects 11 to 15, wherein the target element comprises a portion of a kidney.

Aspect 17. The system of any of Aspects 11 to 16, wherein the machine readable instructions further cause the system to perform, by the processor, pre-processing on at least one image of the first ultrasound image and second ultrasound image, the preprocessing including one or more of a greyscale conversion, blurring, spatial frequency filtering, a histogram equalization, a background subtraction, a contrast enhancement, a brightness normalization, filtering and noise reduction, thresholding, background subtraction.

Aspect 18. The system of any of Aspects 11 to 17, wherein to identify the reference region the machine readable instructions cause the system to receive, via a user interface, a user identification of the reference region.

Aspect 19. The system of any of Aspects 11 to 18, wherein to identify the reference region the machine readable instructions cause the system to identify the reference region via image segmentation.

Aspect 20. The system of any of Aspects 11 to 19, wherein to identify the one or more regions of interest the machine readable instructions cause the system to receive, via a user interface, a user identification of the one or more regions of interest.

Aspect 21. The system of any of Aspects 11 to 20, wherein to identify the reference region the machine readable instructions cause the system to identify, via the processor, the one or more regions of interest via image segmentation

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the target matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion such as a Contrast Agent Injection System shown in FIG. 2) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed:

1. A method for performing ultrasound tracking to determine renal functionality, the method comprising:
obtaining, by an ultrasound sensor, a plurality of ultrasound images of a region of tissue of a patient, the plurality of images including a first ultrasound image and a second ultrasound image, the second ultrasound image obtained at a different time than the first ultrasound image;
identifying a reference region at a first position in the first ultrasound image, the reference region including a reference element;
identifying, one or more regions of interest in the first ultrasound image, the one or more regions of interest including one or more target elements, the one or more regions of interest each having respective first positions defined relative to the reference region in the first ultrasound image;
determining, by the processor, a second position of the reference region in the second image of the plurality of images;
determining, by the processor, a change in position of the reference region from the first position and the second position of the reference region;
determining, by the processor, a second position of each of the regions of interest in the second ultrasound image from the change in position of the reference region;
determining, by the processor, respective ultrasound signal intensity values for each of the one or more regions of interest in the first and second ultrasound images;
generating, via the processor, a time dependent ultrasound signal intensity curve from the respective ultrasound signal intensity values; and,
determining renal functionality from the time dependent ultrasound signal intensity curve.

2. The method of claim 1, further comprising filtering, by the processor, the signal intensity values for each of the one or more regions of interest according to one or more intensity thresholds and generating a filtered set of ultrasound signal intensity values, and wherein generating the time dependent ultrasound signal intensity curve comprises determining the time dependent ultrasound intensity curve from the filtered set of ultrasound signal intensity values.

3. The method of claim 1, wherein determining the renal function from the time dependent ultrasound signal intensity curve comprises determining one or more of a decay time of the curve, mean transient time (MTT), time to peak (TPP), full width at half maximum (FWHM), peak intensity (PI), area under the curve (AUC), wash in time (WIN), and wash out time (WOT).

4. The method of claim 1, further comprising:
averaging, by the processor, the determined ultrasound signal intensity values to generate an averaged time dependent ultrasound signal intensity curve, and;
determining, by the processor, the renal functionality from the averaged time dependent ultrasound signal intensity curve.

5. The method of claim 1, wherein the reference element comprises a portion of a kidney.

6. The method of claim 1, wherein the target element comprises a portion of a kidney.

7. The method of claim 1, further comprising performing, by the processor, pre-processing on at least one image of the first ultrasound image and second ultrasound image, the preprocessing including one or more of a greyscale conversion, blurring, spatial frequency filtering, a histogram equalization, a background subtraction, a contrast enhancement, a brightness normalization, filtering and noise reduction, and thresholding.

8. The method of claim 1, wherein identifying the reference region comprises receiving, via a user interface, a user identification of the reference region.

9. The method of claim 1, wherein identifying the reference region comprises identifying, via the processor, the reference region via image segmentation.

10. The method of claim 1, wherein identifying the one or more regions of interest comprises receiving, via a user interface, a user identification of the one or more regions of interest.

11. The method of claim 1, wherein identifying the reference region comprises identifying, via the processor, the one or more regions of interest via image segmentation.

12. A system for performing ultrasound tracking, the system comprising:
an ultrasound detector;
a processor configured to execute machine readable instructions; and
a non-transitory computer-readable memory having machine readable instructions stored thereon, that when executed by the processor, cause the system to:
obtain, by an ultrasound sensor, a plurality of ultrasound images of a region of tissue of a patient, the plurality of images including a first ultrasound image and a second ultrasound image, the second ultrasound image obtained at a different time than the first ultrasound image;
identify a reference region at a first position in the first ultrasound image, the reference region including a reference element;
identify one or more regions of interest in the first ultrasound image, the one or more regions of interest including one or more target elements, the one or more regions of interest each having respective first positions relative to the reference region in the first ultrasound image;
determine a second position of the reference region in the second image of the plurality of images;
determine a change in position of the reference region from the first position and the second position of the reference region;
determine a second position of each of the regions of interest in the second ultrasound image from the change in position of the reference region;
determine respective ultrasound signal intensity values for each of the one or more regions of interest in the first and second ultrasound images;
generate a time dependent ultrasound signal intensity curve from the respective ultrasound signal intensity values; and,
determine renal functionality and evaluate pyeloplasty from the time dependent ultrasound signal intensity curve.

12. The system of claim 11, wherein the machine readable instructions further cause the system to filter the signal intensity values for each of the one or more regions of interest according to one or more intensity thresholds and generate a filtered set of ultrasound signal intensity values, and wherein to generate the time dependent ultrasound signal intensity curve, the machine readable instructions cause the system to determine the time dependent ultrasound intensity curve from the filtered set of ultrasound signal intensity values.

13. The system of claim 11, wherein to determine the renal function from the time dependent ultrasound signal intensity curve, the machine readable instructions cause the system to determine one or more of a decay time of the curve, mean transient time (MTT), time to peak (TPP), full width at half maximum (FWHM), peak intensity (PI), area under the curve (AUC), wash in time (WIN), and wash out time (WOT).

14. The system of claim 11, wherein the machine readable instructions further cause the system to:
average the determined ultrasound signal intensity values to generate an averaged time dependent ultrasound signal intensity curve, and;
determine the renal functionality from the averaged time dependent ultrasound signal intensity curve.

15. The system of claim 1, wherein the reference element comprises a portion of a kidney.

16. The system of claim 11, wherein the target element comprises a portion of a kidney.

17. The system of claim 11, wherein the machine readable instructions further cause the system to perform, by the processor, pre-processing on at least one image of the first ultrasound image and second ultrasound image, the preprocessing including one or more of a greyscale conversion, blurring, spatial frequency filtering, a histogram equalization, a background subtraction, a contrast enhancement, a brightness normalization, filtering and noise reduction, and thresholding.

18. The system of claim 11, wherein to identify the reference region the machine readable instructions cause the system to receive, via a user interface, a user identification of the reference region.

19. The system of claim 11, wherein to identify the reference region the machine readable instructions cause the system to identify the reference region via image segmentation.

20. The system of claim 11, wherein to identify the one or more regions of interest the machine readable instructions cause the system to receive, via a user interface, a user identification of the one or more regions of interest.

21. The system of claim 11, wherein to identify the reference region the machine readable instructions cause the system to identify, via the processor, the one or more regions of interest via image segmentation.
